(19) United States
(12) Patent Application Publication
(10) Pub. No.: US 2022/0372510 A1
(43) Pub. Date: Nov. 24, 2022

(54) RECOMBINANT SEQUENCE SPECIFICALLY EXPRESSING HUMAN BETA-GLOBIN IN ERYTHROID CELLS, AND USE THEREOF

(71) Applicant: Guizhou Medical University, Guiyang City (CN)

(72) Inventors: Peng ZHANG, Guiyang City (CN); Honglan YANG, Guiyang City (CN); Zhixu HE, Guiyang City (CN); Han LIU, Guiyang City (CN); Yanhua ZHOU, Guiyang City (CN); Anran FAN, Guiyang City (CN)

(21) Appl. No.: 17/726,347

(22) Filed: Apr. 21, 2022

(30) Foreign Application Priority Data
Apr. 21, 2021 (CN) 202110428566.4

(51) Int. Cl. C12N 15/85; C07K 14/805; C12N 15/86; A61K 48/00; A61P 7/06

(52) U.S. Cl. CPC C12N 15/85; C07K 14/805; C12N 15/86; A61K 48/0058; A61P 7/06; A61K 38/00

(57) ABSTRACT

Disclosed is an erythroid-specific human β-globin gene promoter and a human β-globin-expressing recombinant sequence, and use thereof, which belongs to the technical field of genetic engineering. An erythroid-specific human β-globin gene promoter with a nucleotide sequence set forth in SEQ ID NO: 1 is provided in the present disclosure, which may achieve high-efficiency and erythroid-specific initiation of the expression of a functional gene. A recombinant sequence specifically expressing human β-globin in erythroid cells is also provided in the present disclosure, which is human β-globin locus control region (LCR) HS 3-1+β-globin gene promoter+β-globin gene+β-globin gene enhancer.

### CROSS REFERENCE TO RELATED APPLICATION

This disclosure claims the benefit and priority of Chinese Patent Application No. 202110428566.4, entitled “RECOMBINANT SEQUENCE SPECIFICALLY EXPRESSING HUMAN β-GLOBIN IN ERYTHROID CELLS, AND USE THEREOF” filed on Apr. 21, 2021, the disclosure of which is incorporated by reference herein in its entirety as part of the present application.

### TECHNICAL FIELD

The present disclosure belongs to the technical field of genetic engineering, and in particular to a recombinant sequence specifically expressing human β-globin in erythroid cells, and use thereof.

### BACKGROUND ART

β-thalassemia is a hematologic disease caused by a mutation of a gene cluster encoding β-globin. Mediterranean Sea, Africa, Southeast Asia, the Indian Continent, and Southwest and South China are high incidence area of β-thalassemia. In china, Guangxi, Guangdong, Fujian, Taiwan, Hong Kong, Yunnan, Guizhou, and Hainan and other provinces are high incidence regions of β-thalassemia. Thalassemia is a common genetic disease that not only affects the life and health of a patient, but also affects the health of offspring of the patient.

β-thalassemia is caused by a mutation of a β-globin gene cluster located at sub-band 4 of band 5 of short arm region 1 of chromosome 11 (11p15.4). The β-globin gene cluster includes five structural genes, namely β, δ, Aγ, Gγ, and E genes, and hemoglobins (Hbs) encoded by these genes are HBB, HBD, HBG1, HBG2, and HBE, respectively. During the development of human Hb, these structural genes are not continuously expressed, and there is a conversion process of Hb. In a fetal period, the HBG1 and HBG2 genes are mainly expressed to encode γ-globin chains. After birth, the HBB gene is expressed to encode β-globin chains, and the γ and β chains polymerize with an a chain encoded by the α globin gene to form Hb, which delivers oxygen ultimately to various organs throughout the body. Normally, the amounts of β-globin chain and α-globin chain in the body may be consistent. When the HBB gene encoding the β-globin chain is mutated, the corresponding synthesis of β chain will be reduced or absent, resulting in the reduction or absence of synthesis of human HbA (tetramer of α2β2) in adults, intracellular deposition of a chains, cell destabilization, premature destruction of erythroid precursors in bone marrow, shortened lifespan of mature erythrocytes, primer hemolysis, chronic hemolysis, iron overload, hepatosplenomegaly, extramedullary hematopoiesis (EMH), skeletal dysplasia, and the like, and may even be complicated by cardiac, hepatic, and endocrine dysfunctions in severe cases.

At present, the clinical methods for treating β-thalassemia in children mainly include long-term high-volume blood transfusion in combination with standardized iron chelation therapy (ICT). Children with thalassemia major, if not treated, mostly die before the age of 5. β-thalassemia seriously threatens the health of children with β-thalassemia and their families. Some patients with thalassemia intermedia and major require long-term red blood cell transfusion (RBCT) to survive and prevent serious complications, but frequent blood transfusion can easily lead to heart failure and iron overload, and iron deposition may cause functional impairment to multiple organs and may even cause multiple-organ failure (MOF). In particular, children with β-thalassemia major rely on long-term blood transfusion to correct severe anemia. However, only a small number of patients can adhere to standardized treatment, and long-term treatment brings a heavy burden to the family and society. Allogeneic hematopoietic stem cell transplantation (allo-HSCT) is currently the only cure for β-thalassemia, and it has been reported that a cure rate of allo-HSCT in the treatment of thalassemia can reach 80% or higher, and can even reach 90% in some cases. However, it is reported that only less than 30% of children with thalassemia can acquire a suitable matched donor. Hydroxyurea (HU), as a DNA replication inhibitor, increases the expression of normal fetal hemoglobin (HbF) in patients with sickle cell anemia and thalassemia, thereby partially replacing abnormal β-globin. However, this treatment does not work well in all patients, and HU is toxic to some extent. Therefore, it is necessary to seek an efficient and safe treatment for thalassemia, such as to benefit an increased number of children with thalassemia.

In recent years, the gene therapy of β-thalassemia based on β-globin gene addition has been investigated. In 2000, a mouse model for treating β-thalassemia through β-globin gene addition was successfully established, but the research on β-thalassemia treatment by β-globin gene addition was stagnant due to the insufficient effectiveness and safety of a vector. In 2006, a French clinical trial LG001 used HPV569 as a vector for bone marrow hematopoietic stem cell (HSC) editing and transplantation to treat a patient with β0/β+ thalassemia, which benefited the patient and stopped blood transfusion. Subsequently, experimental studies and phase I and II clinical trials on lentiviral vector-based β-globin gene addition were started in many centers. The GLOBE vector-mediated somatic CD34⁺ cell transplantation conducted in Italy in 2016 also achieved a prominent therapeutic effect. Until 2018, a breakthrough was made for the transduction of HSCs with a BB305 lentiviral vector to cure β-thalassemia patients reported on the New England Journal of Medicine (NEJM), which allowed researchers to recognize once again that the viral vector-based β-globin gene addition strategy may become a new means for curing children with β-thalassemia. In 2018, it was reported on the New England Journal of Medicine (NEJM) that, in clinical trials HGB-204 and HGB-205 in the United States, France, Australia, and Thailand since 2013, autologous CD34⁺ cell transplantation mediated by BB305 vector had cured 68% (15/22) of patients with β-thalassemia major, the blood transfusion requirements of the remaining 7 patients decreased to varying degrees, and no serious adverse events related to drug products occurred during 5-year follow-up. The above clinical studies of multiple centers show that more and more β-thalassemia patients can benefit from the gene therapy based on β-globin gene addition, indicating that the lentiviral vector-based β-globin gene addition and HSC transplantation therapeutic strategy is expected to become a new way for curing β-thalassemia. However, because the regulation of β-globin expression is very complicated, how to obtain a high-efficiency β-globin expression sequence in vitro is crucial for the gene therapy of β-thalassemia.

### SUMMARY

In view of this, an objective of the present disclosure is to provide an erythroid-specific human β-globin gene promoter, which can efficiently and specifically initiate the expression of a gene encoding human β-globin in erythroid cells.

Another objective of the present disclosure is to provide a recombinant sequence specifically expressing human β-globin in erythroid cells and use thereof. The recombinant sequence can efficiently express β-globin in erythroid cells and can be used to construct a lentiviral vector for treating pi-thalassemia, thereby effectively improving a success rate of treating β-thalassemia by gene addition.

The present disclosure provides an erythroid-specific human β-globin gene promoter with a nucleotide sequence set forth in SEQ ID NO: 1.

The present disclosure provides a recombinant sequence specifically expressing human β-globin in erythroid cells, wherein the recombinant sequence is human β-globin locus control region (LCR) HS 3-1+human β-globin gene promoter+β-globin gene+β-globin gene enhancer; and

the human β-globin gene promoter is an erythroid-specific human β-globin gene promoter.

In some embodiments, a nucleotide sequence of the β-globin gene enhancer is set forth in SEQ ID NO: 2.

In some embodiments, a nucleotide sequence of the β-globin gene is set forth in SEQ ID NO: 3.

In some embodiments, the β-globin LCR HS 3-1 may include β-globin LCR HS3, β-globin LCR HS2, and β-globin LCR HS1 in sequence;

a nucleotide sequence of the β-globin LCR HS1 is set forth in SEQ ID NO: 4;

a nucleotide sequence of the β-globin LCR HS2 is set forth in SEQ ID NO: 5; and

a nucleotide sequence of the β-globin LCR HS3 is set forth in SEQ ID NO: 6.

In some embodiments, the recombinant sequence may include at least one selected from the group consisting of the following DNA sequences:

1) a DNA sequence with a nucleotide sequence set forth in SEQ ID NO: 7; and

2) a DNA sequence encoding a recombinant protein with an amino acid sequence set forth in SEQ ID NO: 8.

The present disclosure provides a recombinant vector including the recombinant sequence.

In some embodiments, a backbone vector for the recombinant sequence may include a mammalian expression vector and a lentiviral vector.

The present disclosure provides use of the erythroid-specific human β-globin gene promoter, the recombinant sequence, or the recombinant vector in the preparation of a drug for gene therapy of β-thalassemia.

The present disclosure provides a drug for gene therapy of β-thalassemia, including the erythroid-specific human β-globin gene promoter, the recombinant sequence, or the recombinant vector.

The present disclosure provides a human β-globin gene promoter that specifically activates the expression of a functional gene in erythroid cells, and a nucleotide sequence of the promoter is set forth in SEQ ID NO: 1. In the present disclosure, based on the reported pathogenesis and gene therapy methods of thalassemia, several promoter sequences with different lengths for β-globin are predicted by bioinformatics, the fragments of 152 bp, 266 bp, and 422 bp before exon 1 for β-globin are selected as promoter sequences, the promoter sequences are each ligated to a vector expressing an enhanced green fluorescent protein (EGFP) sequence to construct a recombinant vector, and the specificity and effectiveness of the recombinant vector are verified in erythroid cells and other cells. Results show that an expression level of the fluorescent protein (FP) in K562 cells containing a promoter with a length of 266 bp is significantly higher than that in other groups, and the expression levels in the other groups are extremely low, indicating that the promoter set forth in SEQ ID NO: 1 can not only efficiently initiate the expression of a functional gene, but also specifically express the target gene in erythroid cells.

The present disclosure also provides a recombinant sequence specifically expressing human β-globin in erythroid cells, which is human β-globin LCR HS 3-1+human β-globin gene promoter+β-globin gene+β-globin gene enhancer; and the human β-globin gene promoter is an erythroid-specific human β-globin gene promoter. In the present disclosure, the promoter is utilized to initiate the expression of the functional gene in vitro; and the functional gene is recombined with a mammalian expression vector, the specificity is verified in different cell lines, the comparative study on expression efficiency is conducted in erythroid cells, and then different regulatory sequences for β-globin are amplified in vitro. The HS1 sequence is introduced for the first time, different combinations of β-globin regulatory sequences are subjected to comparative study in terms of regulation efficiency for the first time, the regulatory sequence is optimized and recombined with the obtained β-globin expression sequence, and the expression efficiency is verified in erythroid cells, such as to finally obtain a recombinant sequence that efficiently and specifically expresses the human β-globin. The recombinant sequence obtained by the present disclosure can specifically and efficiently express pi-globin in erythroid cells, which provides a basis and experimental reference for gene therapy of β-thalassemia, is of great clinical significance for the treatment of β-thalassemia, and provides a method support for the study of expression regulation of β-globin.

### BRIEF DESCRIPTION OF THE DRAWINGS

FIGS. 1A-B show the electrophoresis results of PCR products of promoters with different lengths and ligated plasmids subjected to enzyme digestion, FIG. 1A shows the electrophoresis results of PCR products of promoters with lengths of 152 bp, 266 bp, and 422 bp, corresponding to 8005 plasmid, 8004 plasmid, and 8006 plasmid, respectively; and FIG. 1B shows the double enzyme digestion identification results of the constructed promoter plasmids with different lengths;

FIG. 2 shows the sequencing and alignment results of the 8004 plasmid;

FIGS. 3A-B show the viability detection results of K562 cells transfected with promoter plasmids with different lengths, where FIG. 3A shows the 12-h detection results; and FIG. 3B shows the 24-h detection results;

FIG. 4 shows the specificity detection results (24 h) in different cells transfected with the 8004 promoter plasmid;

FIGS. 5A-D show the detection results of β-globin expression in K562 cells into which the 8019 plasmid is constructed and transfected, where FIG. 5A is an electropherogram of the 8019 plasmid subjected to double digestion with AseI and NotI; FIG. 5B is a conclusion of sequencing and alignment results of the 8019 plasmid; FIG. 5C shows the detection results of β-globin mRNA in K562 cells electroporated with the 8019 plasmid; and FIG. 5D shows the detection results of β-globin in K562 cells electroporated with the 8019 plasmid;

FIGS. 6A-C show the construction of reverse β-globin plasmids with different HSs and the comparison results of effectiveness in K562 cells, where FIG. 6A shows the electrophoresis results of the 8023, 8405, and 8406 plasmids subjected to KpnI digestion; FIG. 6B shows the Western blotting (WB) detection results of β-globin in K562 cells electroporated with the 8023, 8405, and 8406 plasmids; and FIG. 6C is a conclusion of WB detection results of β-globin in K562 cells electroporated with the 8023, 8405, and 8406 plasmids;

FIG. 7 shows the sequencing and alignment results of the 8405 plasmid fragments; and

FIG. 8 shows the preliminary verification results of specificity of the 8023 recombinant plasmid in different cells.

### DETAILED DESCRIPTION OF THE EMBODIMENTS

The present disclosure provides an erythroid-specific human β-globin gene promoter with a nucleotide sequence set forth in SEQ ID NO: 1

(ATCGTAAATACACTTGCAAAGGAGGATGTTTTTAGTAGCAATTTGTACTG
ATGGTATGGGGCCAAGAGATATATCTTAGAGGGAGGGCTGAGGGTTTGAAG
TCCAACTCCTAAGCCAGTGCCAGAAGAGCCAAGGACAGGTACGGCTGTCAT
CACTTAGACCTCACCCTGTGGAGCCACACCCTAGGGTTGGCCAATCTACTC
CCAGGAGCAGGGAGGGCAGGAGCCAGGGCTGGGCATAAAAGTCAGGGCAGA
GCCATCTATTGCTT).

In the present disclosure, human fi-globin gene promoters with different lengths are predicted by software, and recombinant plasmids containing promoters of three different lengths (152 bp, 266 bp, and 422 bp) are constructed. Results show that the promoter with a length of 266 bp can specifically and efficiently initiate the expression of the target gene in erythroid cells, and the expression levels of the target gene initiated by the other two promoters are low, with a significant difference.

The present disclosure provides a recombinant sequence specifically expressing human β-globin in erythroid cells, which is human β-globin LCR HS 3-1+human β-globin gene promoter+β-globin gene+β-globin gene enhancer; and the human β-globin gene promoter is the erythroid-specific human β-globin gene promoter. In the present disclosure, a nucleotide sequence of the β-globin gene enhancer is preferably set forth in SEQ ID NO: 2. A nucleotide sequence of the β-globin gene is preferably set forth in SEQ ID NO: 3. The β-globin LCR HS 3-1 may preferably include β-globin LCR HS3, β-globin LCR HS2, and β-globin LCR HS1 in sequence; a nucleotide sequence of the β-globin LCR HS1 is preferably set forth in SEQ ID NO: 4; a nucleotide sequence of the β-globin LCR HS2 is preferably set forth in SEQ ID NO: 5; and a nucleotide sequence of the β-globin LCR HS3 is preferably set forth in SEQ ID NO: 6. The recombinant sequence may include at least one selected from the group consisting of the following DNA sequences: 1) a DNA sequence with a nucleotide sequence set forth in SEQ ID NO: 7; and 2) a DNA sequence encoding a recombinant protein with an amino acid sequence set forth in SEQ ID NO: 8. There is no particular limitation on the source of the recombinant sequence in the present disclosure, and a recombinant sequence source well known in the art will do, such as a synthetic or PCR amplified sequence.

In the present disclosure, different regulatory regions are selected to express the human β-globin, and results show that, compared with the regulatory region HS 1-4, the regulatory region HS 1-3 without HS 4 enables the highest expression of β-globin by the β-globin plasmid. Therefore, the regulatory region HS 1-3 without HS 4 is selected as a part of the recombinant sequence for efficient regulation of the expression of human β-globin.

The present disclosure provides a recombinant vector including the recombinant sequence. A backbone vector of the recombinant vector may preferably include a mammalian expression vector and a lentiviral vector. The mammalian expression vector may preferably include pEGFP-N1 and pUC57 vectors. The lentiviral vector may preferably include a pLV-EF1a-OCT4-IRES-eGFP vector. There is no particular limitation on the sources of the mammalian expression vector and the lentiviral vector in the present disclosure, and vector sources well known in the art will do.

The present disclosure provides use of the erythroid-specific human β-globin gene promoter, the recombinant sequence, or the recombinant vector in the preparation of a drug for gene therapy of β-thalassemia.

The present disclosure also provides use of the erythroid-specific human β-globin gene promoter, the recombinant sequence, or the recombinant vector in the gene therapy of β-thalassemia.

In the present disclosure, a method for the gene therapy of β-thalassemia may preferably include the following steps: inserting the recombinant sequence into a mammalian expression vector to obtain a mammalian recombinant expression vector, or inserting the recombinant sequence into a lentiviral vector to obtain a recombinant lentiviral vector, and introducing the mammalian recombinant expression vector or the recombinant lentiviral vector into a patient to realize the recombinant expression of β-globin in the patient, which increases an expression level of β-globin to achieve the purpose of treating β-thalassemia.

The present disclosure provides a drug for gene therapy of β-thalassemia, including the erythroid-specific human β-globin gene promoter, the recombinant sequence, or the recombinant vector. The drug may preferably further include a pharmaceutically acceptable adjuvant. The drug achieves the purpose of treating β-thalassemia through the recombinant expression of the recombinant sequence in cells of a patient to increase a content of β-globin.

The recombinant sequence specifically expressing human β-globin in erythroid cells and use thereof provided by the present disclosure are described in detail below with reference to examples, but these examples may not be understood as a limitation to the protection scope of the present disclosure.

### Example 1

Construction of Promoter Plasmids with Different Lengths and Determination of a Promoter with the Optimal Activity

1. A promoter core region for β-globin was predicted on the website http://www.genomatix.de/, and in combination with the promoter lengths in different vectors described in queried literatures, the fragments of 152 bp, 266 bp, and 422 bp before exon 1 for β-globin were finally selected.

2. A single restriction site was selected upstream and downstream of a promoter of a pEGFP-N1 backbone plasmid, with Vsp I upstream and NheI downstream. A forward primer (FP) and a reverse primer (RP) were designed according to an NCBI gene sequence and a predicted promoter region, and a restriction site and a protective base were added at the 5′ end to synthesize primer sequences. Exon-RP was adopted as a common reverse primer. Details were shown in Table 1.

TABLE 1
Primers for amplification of the three
promoter fragments

| Primer | | |
|---|---|---|
| name | Sequence | Sequence No. |
| E152 FP | CGATTAATAGTGCCAGAAGAGCCAAGGA | SEQ ID NO: 9 |
| E266 FP | CGATTAATCGTAAATACACTTGCAAAGG | SEQ ID NO: 10 |
| | AGG | |
| E422 FP | CGATTAATCTGGAGACGCAGGAAGAGAT | SEQ ID NO: 11 |
| Exon-RP | ATATTGCTAGCAAGCAATAGATGGCTCT | SEQ ID NO: 12 |
| | GCC | |

3. Genomic DNA (gDNA) (Item No.: DP304) was extracted from normal human blood, and specific steps were as follows:

1) 200 μL of blood was taken and centrifuged at 3,000 rpm for 5 min, and a resulting supernatant was discarded;

2) 200 μL of Buffer GA was added, and a resulting mixture was pipetted up and down for mixing;

3) 20 μL of Proteinase K was added, and a resulting mixture was thoroughly mixed by inverting a tube up and down;

4) 200 μL of Buffer GB was added, a resulting mixture was thoroughly mixed by inverting the tube up and down and then incubated in a 70° C. metal bath for 10 min, and after a clear solution was obtained, the solution was centrifuged briefly;

5) 200 μL of absolute ethanol was added, and a resulting mixture was thoroughly shaken for 15 s and then centrifuged briefly;

6) the above solution was transferred to a CB3 adsorption column marked in advance, centrifugation was conducted at 12,000 rpm for 1 min, and a resulting filtrate was discarded;

7) 500 μL of Buffer GD was added, centrifugation was conducted at 12,000 rpm for 1 min, and a resulting filtrate was discarded;

8) 600 μL of Buffer PW was added, centrifugation was conducted at 12,000 rpm for 1 min, and a resulting filtrate was discarded;

9) step 8) was repeated, then centrifugation was conducted at 12,000 rpm for 2 min, a resulting filtrate was discarded, and the adsorption column was transferred to a clean 1.5 ml EP tube marked in advance and air-dried for 5 min to 10 min with a cap open;

10) 100 μL of purified water was added to a center of the adsorption column, incubation was conducted at 55° C. for 5 min, centrifugation was conducted at 12,000 rpm for 2 min, and a resulting liquid in the EP tube was collected.

4. The promoter fragment was subjected to PCR amplification (Q5 enzyme, Item No.: M0491S), and specific steps were as follows:

1) Forward and reverse primers were designed and synthesized according to the target fragment to be amplified, and each diluted to 10 mM for use;

2) An ice box was prepared, gloves were worn during the experimental operation, a 0.2 ml PCR tube was prepared, and different pipette tips were used for different reagents. Sample addition was conducted according to the following principle: The samples were added from large-amount to small-amount; the enzyme was added last, which was taken out just before addition; be marked in advance.

3) PCR reaction system and parameters were shown in Table 2 (the system could be multiplied according to the experimental purpose).

TABLE 2
Reaction system

| Component | Volume | |
|---|---|---|
| 5 × Q5 reaction Buffer | 10 | μL |
| 5 × Q5 GC enhancer | 10 | μL |
| 10 mM dNTP | 1 | μL |
| 10 mM forward primer | 2.5 | μL |
| 10 mM reverse primer | 2.5 | μL |
| DNA template | 0.1-2 | μg |
| Q5 enzyme | 0.5 | μL |
| Making up with ddH₂O to | 50 | μL |

PCR Amplification Procedure:

Step 1 pre-denaturation: 95° C. for 3 min;

Step 2 denaturation: 95° C. for 30 s;

Step 3 annealing: 60° C. for 30 s;

Step 4 extension: 72° C. for 30 s, 40 cycles;

Step 5 re-extension: 72° C. for 5 min.

5. Purification of PCR product (Item No.: DP204-02):

1) column equilibration: 500 μL of buffer BL was added to a CB3 adsorption column, centrifugation was conducted at 12,000 rpm for 1 min, and a resulting filtrate was discarded;

2) 5 folds of buffer PB was added to the PCR product, and a resulting mixture was thoroughly mixed;

3) the mixture obtained in step 2) was transferred to the equilibrated CB3 (which needed to be marked in advance), centrifugation was conducted at 12,000 rpm for 1 min, and a resulting filtrate was discarded;

4) 600 μL of PW was added, the column was allowed to stand for 2 min to 5 min at room temperature, centrifugation was conducted at 12,000 rpm for 1 min, and a resulting filtrate was discarded;

5) step 4) was repeated;

6) centrifugation was conducted at 12,000 rpm for 2 min, a resulting filtrate was discarded, and the column was allowed to stand at room temperature for few minutes to be air-dried; and

7) CB3 was transferred to a new 1.5 mL EP tube marked in advance, 50 μL or more of an eluent was added dropwise in a suspended manner to a center of an adsorption membrane, centrifugation was conducted at 12,000 rpm for 2 min, and a resulting filtrate was collected for concentration determination.

6. VspI and NheI double enzyme digestion backbone and target fragment (Item No.: VspI: FD0914, and NheI: FD0947). An enzyme digestion system was shown in Table 3 (which could be proportionally decreased or increased).

TABLE 3
Enzyme digestion reaction system

| Component | Volume | |
|---|---|---|
| Backbone or target fragment | <2 | μg |
| 10 × Green buffer | 3 | μL |
| Vsp I | 1 | μL |
| Hind III | 1 | μL |
| Making up with ddH₂O to | 30 | μL |

7. A target band obtained after enzyme digestion was recovered by subjecting the PCR product to gel extraction, and a long EGFP-containing fragment among enzyme digestion products of the backbone plasmid was recovered (gel extraction kit, Item No.: K0691). Specific steps were as follows:

1) an electrophoresis tank and the like were cleaned, and 1% agarose gel was prepared;

2) sample loading: 5 μL of Mark was added, and all digestion products were added to sample wells every other well;

3) electrophoresis was conducted at 120 V for 30 min, and a 1.5 mL EP tube was taken, marked, and weighed;

4) gel with the target band was cut and collected in the EP tube;

5) a total weight of the gel and the tube was measured and subtracted by a weight of the tube to obtain a weight of the gel;

6) binding buffer was added to the gel, and a resulting mixture was incubated in a 55° C. metal bath for 10 min until the gel was completely dissolved, where a ratio of the weight of gel to a volume of binding buffer was 1:1;

7) isopropyl alcohol (IPA) was added, and a resulting mixture was pipetted up and down for mixing, where a ratio of the weight of gel to a volume of IPA was 1:1;

8) the mixture was transferred to an adsorption column marked in advance, centrifugation was conducted at 12,000 rpm for 1 min, and a resulting filtrate was discarded; and then 100 μL of binding buffer was added, centrifugation was conducted at 12,000 rpm for 2 min, and a resulting filtrate was discarded;

9) 700 μL of wash buffer was added, centrifugation was conducted at 12,000 rpm for 2 min, and a resulting filtrate was discarded; centrifugation was conducted at 12,000 rpm for 2 min, and a resulting filtrate was discarded; and the column was transferred to a new EP tube marked in advance, and then air-dried for 5 min; and

10) 30 μL of pure water was added, and the mixture was incubated in a 55° C. metal bath for 5 min; and centrifugation was conducted at 12,000 rpm for 2 min, a resulting filtrate was collected, and 2 μL of the filtrate was taken for concentration determination.

8. Ligation (T4 DNA ligase, Item No.: EL0011). Specific steps were as follows:

Fragment:backbone=3:1 (mass ratio), and thus fragment (ng)=backbone (ng)×3×fragment length/backbone fragment length. A specific ligation system was shown in Table 4. After samples were added, the ligation was conducted at room temperature for 2 h or overnight.

TABLE 4
Ligation reaction system

| Component | Volume | |
|---|---|---|
| Backbone | 50-100 | ng |
| Target fragment | 300 ng × fragment | |
| | length/backbone length | |
| T4 DNA Ligase buffer | 1 | μL |
| T4 DNA Ligase | 1 | μL |

| | |
|---|---|
| ddH₂O | Making up to 20 μL |

9. Transformation (Item No.: CB101-02):

1) the plasmid was taken out and put on ice, and competent cells were taken out and thawed on ice;

2) 5 μL of the plasmid was added to 50 μL of the competent cells, and a resulting mixture was allowed to stand on ice for 30 min;

3) heat shock: the heat shock was conducted at 42° C. for 90 s;

4) resulting cells were immediately allowed to stand on ice for 5 min;

5) recovery: 150 μL of a resistance-free liquid medium was added, and the cells were cultivated for 1 h at 37° C. and 160 rpm;

6) coating: a recovered bacterial solution was taken and spread on an LB resistant solid medium by sterilized beads; and

7) cultivation was conducted overnight at 37° C. and the colony growth was observed.

10. Amplification of monoclonal colonies:

a 15 mL centrifuge tube was taken, 5 mL of a resistance-free liquid medium was added, kana was added to make its final concentration 50 μg/mL, and single colonies were picked and added to the centrifuge tube with a high-pressure pipette tip, and the colonies were cultivated overnight at 37° C. and 160 rpm.

11. Extraction of amplified plasmid (Item No.: AP-MN-P-50): Specific steps were as follows:

1) 1 ml to 4 ml of a bacterial culture was taken and centrifuged at 12,000 rpm for 2 min, and a resulting supernatant was discarded (the bacterial culture could be centrifuged multiple times, and a resulting bacterial precipitate was collected in a centrifuge tube).

2) 250 μL of Buffer S1 (if it was used for the first time, RNaseA was added before use) was added to the centrifuge tube with the bacterial precipitate, and a resulting mixture was pipetted up and down and resuspended until there was no small bacterial mass;

3) 250 μL of Buffer S2 was added to the centrifuge tube, and the centrifuge tube was gently inverted up and down for 6 to 8 times to fully lyse the bacteria (notes: the mixing must be conducted gently to avoid contamination to the bacterial gDNA, such that a resulting bacterial solution became clear and viscous; and a time of the mixing should not exceed 5 min, such as to avoid damage to the plasmid);

4) 350 μl of Buffer S3 was added to the centrifuge tube, and the centrifuge tube was immediately gently inverted up and down for 6 to 8 times until a white flocculent precipitate appeared; a resulting mixture was centrifuged at 12,000 rpm for 10 min, and a resulting supernatant was carefully transferred to an adsorption column marked in advance (the adsorption column was added to the collection tube) and placed at room temperature for 2 min; and centrifugation was conducted at 12,000 rpm for 1 min, a resulting filtrate was discarded, and the adsorption column was put back to the collection tube;

5) 500 μL of a washing solution W1 was added to the adsorption column, centrifugation was conducted at 12,000 rpm for 1 min, a resulting filtrate was discarded, and the adsorption column was put back to the collection tube;

6) 700 μL of a washing solution W2 was added to the adsorption column (it was checked whether absolute ethanol had been added before the addition of the washing solution), centrifugation was conducted at 12,000 rpm for 1 min, a resulting filtrate was discarded, and the adsorption column was put back to the collection tube;

7) step 6) was repeated;

8) centrifugation was conducted at 12,000 rpm for 2 min, the adsorption column was transferred to a new EP tube marked in advance, and the tube was placed at room temperature for few minutes with a cap open to remove the residual washing solution in the adsorption column; and

9) 60 μL to 80 μL of ddH₂O was added dropwise in a suspended manner to a center of an adsorption membrane, incubation was conducted at 55° C. for 8 min, centrifugation was conducted at 12,000 rpm for 2 min, and a resulting filtrate was collected, which was the extracted plasmid DNA.

12. Verify of whether the recombinant plasmid was correctly ligated. The recombinant plasmid pEGFP-βp (Exon152 bp) was numbered 8005; the recombinant plasmid pEGFP-βp (Exon266 bp) was numbered 8004; and the recombinant plasmid pEGFP-βp (Exon422 bp) was numbered 8006.

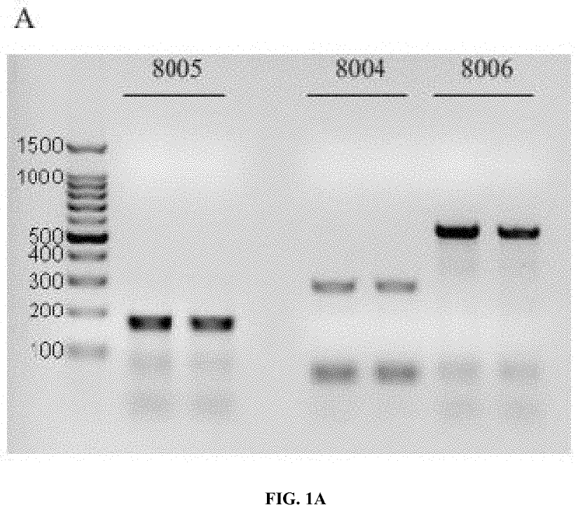

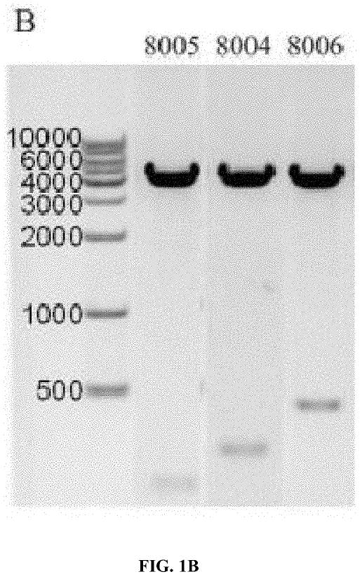

1) Electrophoresis verification after double digestion: An enzyme digestion system was the same as above, and a small system of 10 μL or 5 μL could be used. A band size was verified by electrophoresis after enzyme digestion, as shown in FIG. 1B. The band sizes of the plasmids 8005, 8004, and 8006 after enzyme digestion were 4,148 bp+152 bp, 4,148 bp+266 bp, and 4,148 bp+422 bp, respectively.

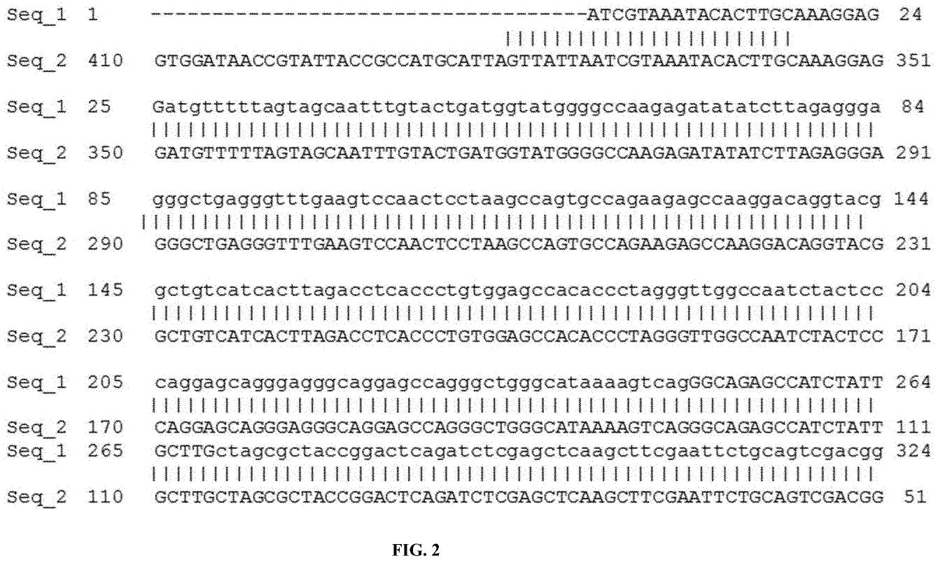

2) Verification by sequencing: The extracted plasmids were sent to a company for sequencing the inserted fragments, such as to further verify whether the plasmids were constructed successfully, and it was determined that the plasmid fragments 8005, 8004, and 8006 were correct. A sequencing result of the plasmid 8004 was shown in FIG. 2.

13. Activity detection and specificity verification for recombinant promoter plasmids:

1) Activity detection for recombinant promoter plasmids with different lengths:

Cell transfection: K562 cells (Item No.: L3000015) were transfected with a liposome. Specific steps were as follows:

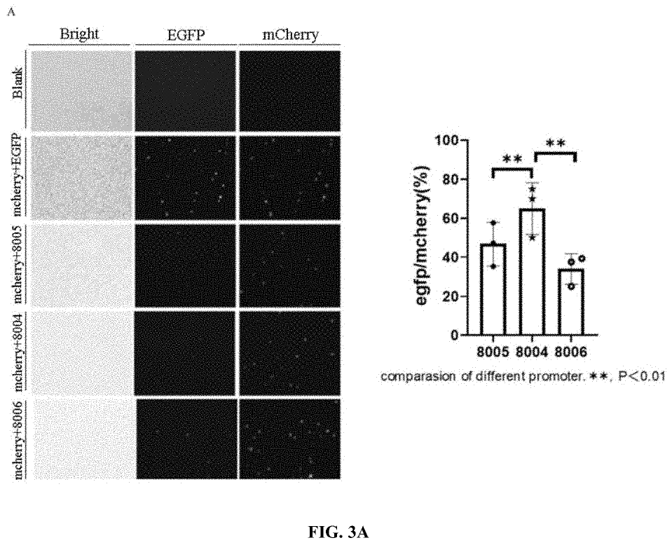

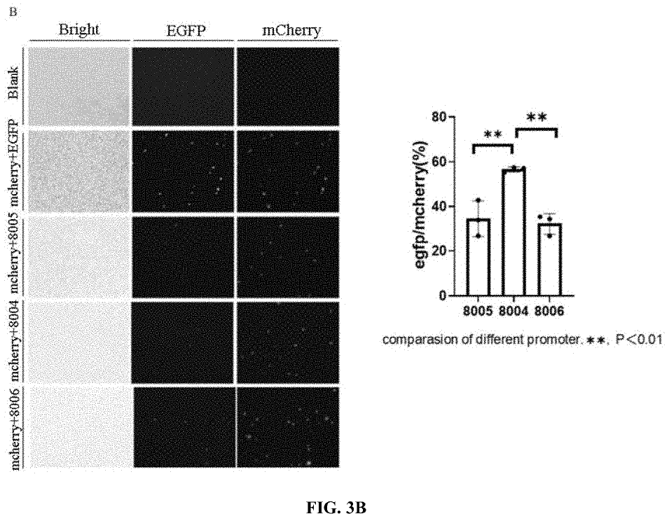

Culture plates were prepared. Cells were collected by centrifugation, washed once with PBS, inoculated into a 6-well plate or a 12-well plate, and the plate was put back to an incubator for continuous culture. Then a transfection system was added: tube A: diluted liposome: 250 μL opti-MEM+7.5 μL lipofection; and tube B: diluted DNA: 250 μL opti-MEM+5 μL P3000+1 μg recombinant plasmid DNA+1 μg mCherry plasmid with red fluorescence. A and B were thoroughly mixed, incubated at room temperature for 15 min, and the incubation solution was added to K562 cell wells, and the cells were cultivated in the incubator. At 12 h and 24 h of the cultivation, the number of cells with green fluorescence in the red fluorescence was observed, and the promoter activity was detected. The activity detection results were shown in FIGS. 3A-B, and it can be seen that, either at 12 h (FIG. 3A) or at 24 h (FIG. 3B), the recombinant plasmid 8005 (that is, when the promoter length was 266 bp) showed the optimal promoter activity.

2) Specificity detection of the recombinant promoter plasmid 8004:

Cell transfection: a liposome was used to transfect different cell lines (Item No.: L3000015), and specific steps were as follows:

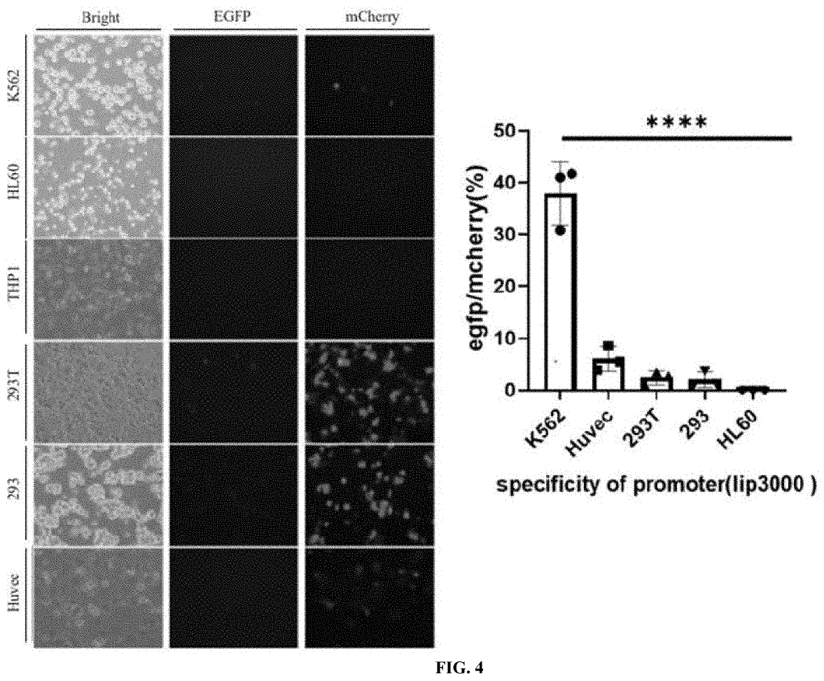

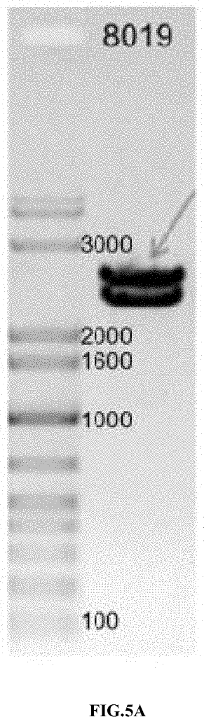

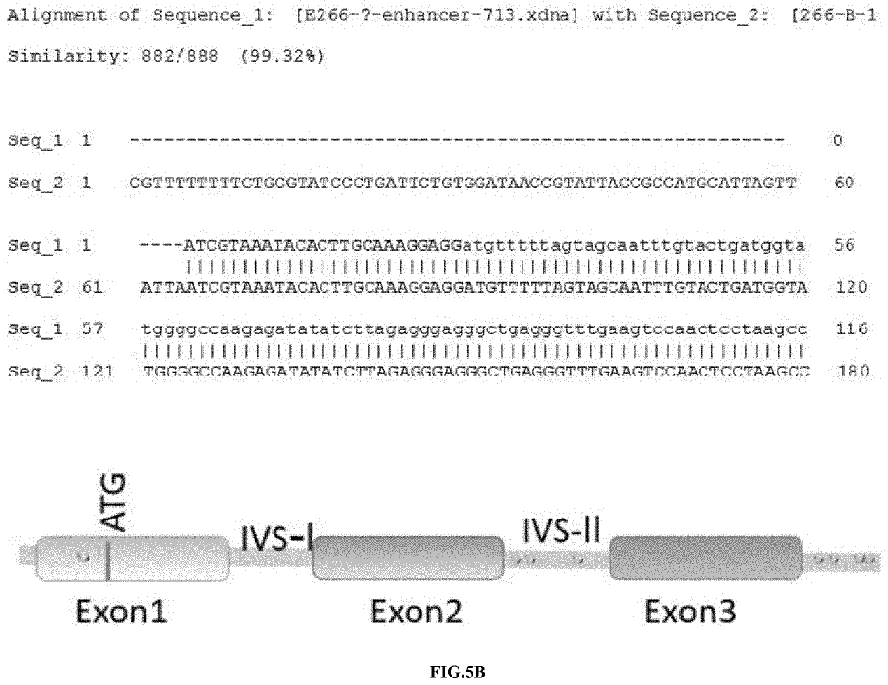

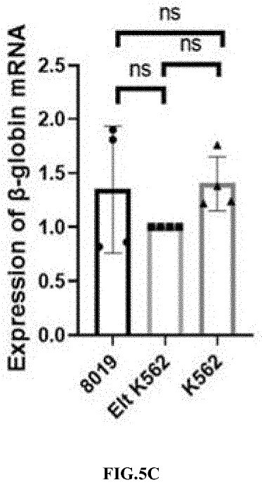

Culture plates were prepared. Cells were collected by centrifugation, washed once with PBS, inoculated into a 6-well plate or a 12-well plate, and the plate was put back to an incubator for continuous culture. Then a transfection system was added: tube A: diluted liposome: 250 μL opti-MEM+7.5 μL lipofection; and tube B: diluted DNA: 250 μL opti-MEM+5 μL P3000+1 μg recombinant plasmid DNA+1 μg mCherry plasmid with red fluorescence. A and B were thoroughly mixed, incubated at room temperature for 15 min, and the incubation solution was added to K562, Huvec, 293T, 293, and HL60 cell wells, and the cells were cultivated in the incubator. At 12 h and 24 h of the cultivation, the number of cells with green fluorescence in the red fluorescence was observed, and the promoter specificity was detected. The specificity detection results were shown in FIG. 4, and it can be seen that an expression level in K562 cells was significantly higher than expression levels in other groups, and the expression levels in the other groups were extremely low.

### Example 2

Construction of a Plasmid without β-Globin LCR (HS) Using the Selected Promoter Length

1. An enhancer core region of β-globin was predicted on the website http://www.genomatix.de/, and in combination with the promoter lengths in different vectors described in queried literatures, a β-globin LCR (HS)-free fragment from the promoter with the optimal activity (266 bp before exon 1) to the β-globin enhancer was amplified, which was named pβp (Exon266 bp)-βglobin-βE-713 and numbered 8019.

2. A single restriction site was selected on the backbone plasmid, with VspI upstream and NotI downstream; and forward and reverse primers were designed according to the NCBI gene sequence and the predicted promoter region, and the restriction site and a protective base were added at the 5′ end.

Table 5 Sequence information of primers for amplification of the promoter protected by the present disclosure

TABLE 5
Sequence information of primers for amplification
of the promoter protected by the present disclosure

| Primer | | |
|---|---|---|
| name | Sequence | Sequence No. |
| E266FP | CGATTAATCGTAAATACACTTGCAA | SEQ ID NO: 10 |
| | AGGAGG | |
| enhancer-R | ATGCGGCCGCTGGTAACACTATGCT | SEQ ID NO: 13 |
| | AATAA | |

3. gDNA was extracted from normal human blood (Item No.: DP304), and specific steps were the same as in Example 1.

4. The β-globin fragment (Phusion enzyme, Item No.: F530S) without the β-globin LCR (HS) was subjected to PCR amplification. A PCR amplification system was shown in Table 6.

TABLE 6
PCR amplification reaction system for β-globin gene fragment

| Component | Volume | |
|---|---|---|
| 5 × HF Buffer | 20 | μL |
| 10 mM dNTP | 2 | μL |
| 10 mM E266FP | 5 | μL |
| 10 mM enhancer-R | 5 | μL |
| DNA template | 0.1-2 | μg |
| Phusion enzyme | 1 | μL |
| Making up with ddH₂O to | 100 | μL |

PCR Amplification Procedure:

Step 1 pre-denaturation: 98° C. for 2 min;

Step 2 denaturation: 98° C. for 10 s;

Step 3 annealing: 63° C. for 30 s;

Step 4 extension: 72° C. for 30 s to 5 min, depending on an amplification length, 40 cycles;

Step 5 re-extension: 72° C. for 5 min;

Step 6 temporary storage: about 30 min at 4° C., depending on a specific time demand.

5. Purification of PCR amplification product (common DNA product purification kit, Item No.: DP-204-02), and specific steps were the same as in Example 1.

6. VspI and NheI double enzyme digestion backbone and target fragment (Item No.: VspI: FD0914, and NotI: FD0594). An enzyme digestion system was shown in Table 7 (which could be proportionally decreased or increased).

TABLE 7
VspI and NheI double enzyme digestion reaction system

| Component | Volume | |
|---|---|---|
| Backbone or target fragment | 2 | μg |
| 10 × Green buffer | 3 | μL |
| VspI | 1 | μL |
| NotI | 1 | μL |
| Making up with ddH₂O to | 30 | μL |

7. The target fragment and 713 plasmid backbone were recovered by gel (gel extraction kit, Item No.: K0691), and specific steps were the same as in Example 1.

8. Ligation (T4 DNA ligase, Item No.: EL0011): Specific steps were the same as in Example 1:

9. Transformation (Item No.: CB101-02): Specific steps were the same as in Example 1.

10. Amplification of monoclonal colonies: Specific steps were the same as in Example 1.

11. Extraction of amplified plasmid (Item No.: AP-MN-P-50): Specific steps were the same as in Example 1.

12. Verify of whether the LCR-free β-globin recombinant plasmid was correctly ligated. The recombinant plasmid was named pβp(Exon266 bp)-βglobin-βE-713 and numbered 8019.

1) Electrophoresis verification after double digestion: An enzyme digestion system was the same as above, and a small system of 10 μL or 5 μL could be used. A band size was verified by electrophoresis after enzyme digestion, as shown in FIG. 5A. The band sizes of the plasmid 8019 after enzyme digestion were 2,718 bp and 3,339 bp.

2) Verification by sequencing: The extracted plasmid was sent to a company for sequencing the inserted fragment, such as to further verify whether the plasmid was constructed successfully. As shown in FIG. 5B, the sequencing result showed that there were 8 sites inconsistent with the NCBI sequence, but these sites were all outside the open reading frame (ORF).

### Example 3

The effectiveness test of the β-globin recombinant plasmid without LCR, K562 cells were electrotransfected with the recombinant plasmid, and untransfected cells and blank transfected cells were set as control groups. A relative expression level of β-globin mRNA was detected by qPCR, and an expression level of β-globin was detected by WB. Results were shown in FIG. 5C and FIG. 5D. Compared with the control groups, the expression of β-globin was not significantly changed with reference to the blank group at either the mRNA level or the protein level, without statistically-significant difference.

(1) K562 cells were electroporated (electrode tube set, Item No.: No. 1207) by the following specific steps:

A culture plate was prepared, a complete medium was added to the culture plate, and the culture plate was incubated in an incubator. Cells were collected by centrifugation and washed once with PBS, 20 μL of an electroporation solution and 4 μg of the recombinant plasmid were added to the cells, and a resulting mixture was pipetted up and down for mixing and then transferred to an electroporation cuvette; then electroporation was conducted according to different electroporation conditions of different cells, and a system obtained after the electroporation was added to a corresponding well of the culture plate incubated in the incubator; and the culture plate was incubated in the incubator for 48 h, then the RNA and protein were extracted, and a content of β-globin was determined.

(2) A specific method for the detection of the relative expression level of β-globin mRNA by qPCR was as follows:

1) RNA extraction (RNA extraction kit, Item No.: R1058): Specific steps were as follows:

A: cells were resuspended and washed once with PBS;

B: cell counting was conducted;

C: a cell precipitate obtained after the washing was collected, 300 μL of a cell lysis solution was added, and a resulting mixture was pipetted up and down for thorough mixing;

D: lysed cells were transferred to a yellow adsorption column, centrifugation was conducted at 12,000 rpm for 2 min, a resulting filtrate was retained, and the adsorption column was discarded;

E: 150 μL of absolute ethanol was added to the filtrate, a resulting mixture was thoroughly mixed and transferred to a green adsorption column, centrifugation was conducted at 12,000 rpm for 2 min, and a resulting filtrate was discarded;

F: 400 μL of wash buffer was added to the adsorption column, centrifugation was conducted, and a resulting filtrate was discarded;

G: an enzyme-free EP tube was taken, 75 μL of DNA digestion buffer and 5 μL of DNase were thoroughly mixed and added to the adsorption column, and then the adsorption column was incubated at room temperature for 15 min;

H: after the incubation was completed, 400 μL of RNAprep buffer was added to the column, centrifugation was conducted at 12,000 rpm for 2 min, and a resulting filtrate was discarded;

I: 700 μL of RNA WASH BUFFER was added to the column, centrifugation was conducted at 12,000 rpm for 2 min, and a resulting filtrate was discarded; and 400 μL of RNA WASH BUFFER was added, centrifugation was conducted at 12,000 rpm for 3 min, and a resulting filtrate was discarded; and

J: an enzyme-free EP tube was taken, the green adsorption column was transferred to the EP tube, 100 μL of DNase/RNase Free Water was added to a center of the column, and centrifugation was conducted at 12,000 rpm for 2 min; an appropriate amount of the obtained RNA was taken out and dispensed for concentration determination and electrophoresis, which was intended to reduce the repeated freezing and thawing of RNA; and the RNA was stored at −80° C. (RNA electrophoresis: an electrophoresis solution was replaced in advance, 100 ng to 500 ng of RNA was taken, thoroughly mixed with loading and enzyme-free water, and then loaded, and electrophoresis was conducted at 10 V/cm)

2) cDNA was synthesized by reverse transcription (reverse transcription kit: Thermo: RevertAid First Strand cDNA Synthesis Kit, Item No. K1621), and specific steps were as follows:

A: a required amount of RNA was calculated. 1 μg of RNA was generally used for a 20 μL system. The system could be appropriately enlarged or reduced (0.1 ng to 5 μg of RNA could be used);

B: reagents other than the enzyme in the reverse transcription kit were taken out and placed on ice;

C: a 0.2 ml enzyme-free EP tube was taken, marked, and placed on ice;

D: reaction preparation (12 μL): RNA 1 μg+Random Primer 1 μL+enzyme-free water (up to 12 μL)

E: the reaction was incubated in a 65° C. metal bath for 5 min, and then put back on ice; and

F: the following reagents were added in sequence: 8 μL in total;

TABLE 8
Reverse transcription reaction

| Component | Volume |
|---|---|
| 5 × Reation Buffer | 4 μL |
| RiboLock RNase Inhibito | 1 μL |
| 10 mM dNTP Mix | 2 μL |
| RevertAid M-MμLV RT (200 U/μL)(enzyme) | 1 μL |

G: brief centrifugation was conducted, and incubation was conducted in a 42° C. metal bath for 60 min;

H: pre-synthesis was conducted at 25° C. for 5 min;

I: a heat treatment was conducted at 70° C. for 5 min to terminate the reaction; and

J: a product was stored at −80° C. or used for subsequent experiments.

3) q-PCR detection of globin level:

q-PCR system: Experimental group: enzyme-free water 6.8 μL+2×SYBR 10 μL+cDNA 1.6 μL+forward primer 0.8 μL+reverse primer 0.8 μL (forward primer: mRNA-F-2: CTGAGGAGAAGTCTGCCGTTA, SEQ ID NO: 14, reverse primer: mRNA-R-2: GAGGTTGTCCAGGTGAGCCA, SEQ ID NO: 15). Blank control group: enzyme-free water 8.4 μL+2×SYBR 10 μL+forward primer 0.8 μL+reverse primer 0.8 μL (3 replicate wells were required for each group, and thus water and cDNA could be mixed in advance and then evenly dispensed to each well).

q-PCR procedure: Details can be seen in the instrument manual. However, an annealing temperature of the primers should be known in advance, an annealing temperature of the primers for the internal reference should not be too different from an annealing temperature of the primers for the experimental group, and an average value is generally taken.

(3) Detection of β-globin level by WB (primary and secondary antibodies for β-globin, Item No.: SC-21757 and 62-6520): Specific steps were as follows:

1) cell collection: cells were collected by centrifugation, washed once with PBS, and counted by a cell counter;

2) total cellular protein (TCP) extraction: a lysis solution (strong lysis solution+PMSF=100:1) was added at an amount enabling 50 μL to 100 μL of lysis solution for 3 to 5×10⁶ cells, and a resulting mixture was shaken on ice for 1 h;

3) protein denaturation: 5×protein loading buffer was added to a protein sample at a final concentration of 1×, and a sample tube was wrapped with a sealing tape and then incubated in a 100° C. metal bath for 10 min;

4) a centrifuge was pre-cooled, then centrifugation was conducted at 12,000 rpm and 4° C. for 20 min, and a resulting supernatant was collected, which was the protein sample;

5) gel preparation;

6) the supernatant obtained after the centrifugation was taken and loaded for electrophoresis;

7) transfer and blocking;

8) primary antibody incubation: the membrane was incubated for 1 h at room temperature or overnight at 4° C., and then washed with PBST three times, each time for 10 min;

9) secondary antibody incubation: the membrane was incubated for 1 h at room temperature or overnight at 4° C., and then washed with PBST three times, each time for 10 min; and

10) an exposure solution was prepared according to a ratio of 1:1 and then added dropwise onto the washed membrane, and exposure was conducted with a Bio-rad exposure machine.

### Example 4

With the screened promoter length, reverse β-globin plasmids with different β-globin LCRs (HS) were constructed, the effectiveness and specificity of which were tested, and an efficient and specific recombinant sequence carrying human β-globin gene was finally obtained.

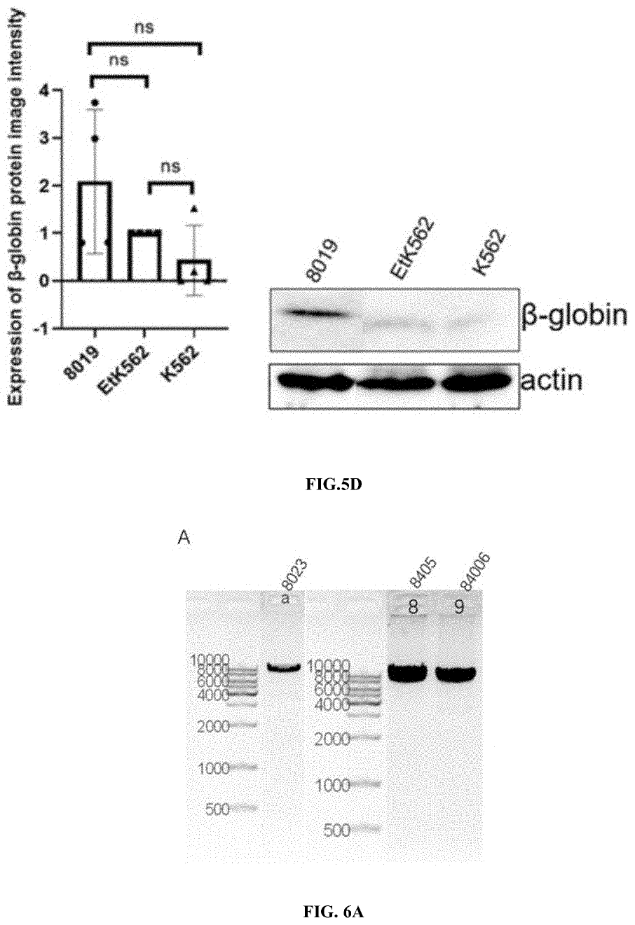

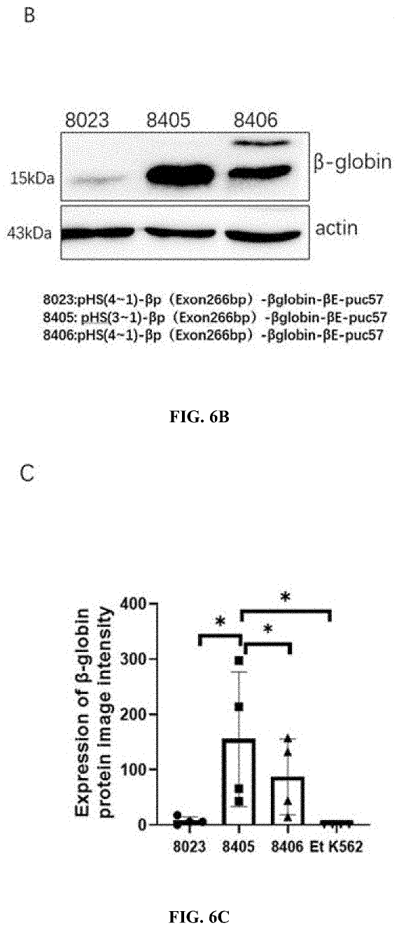

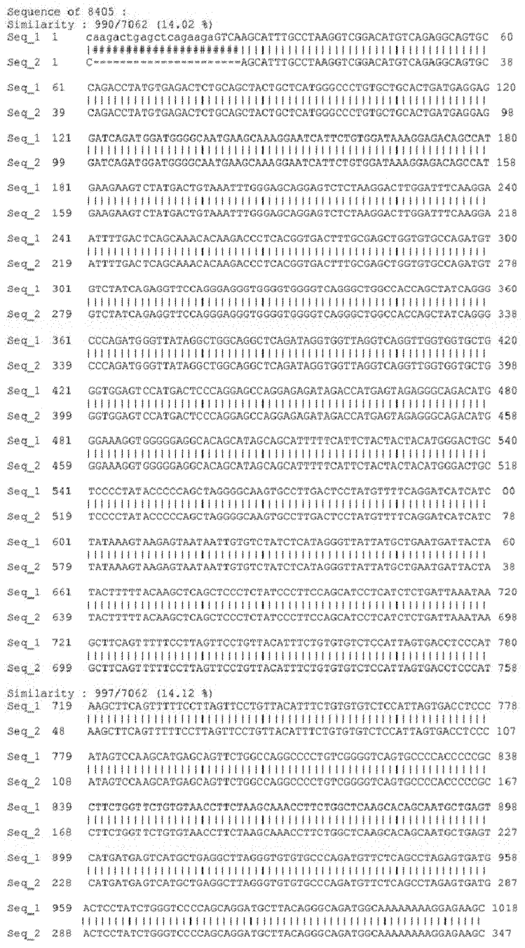

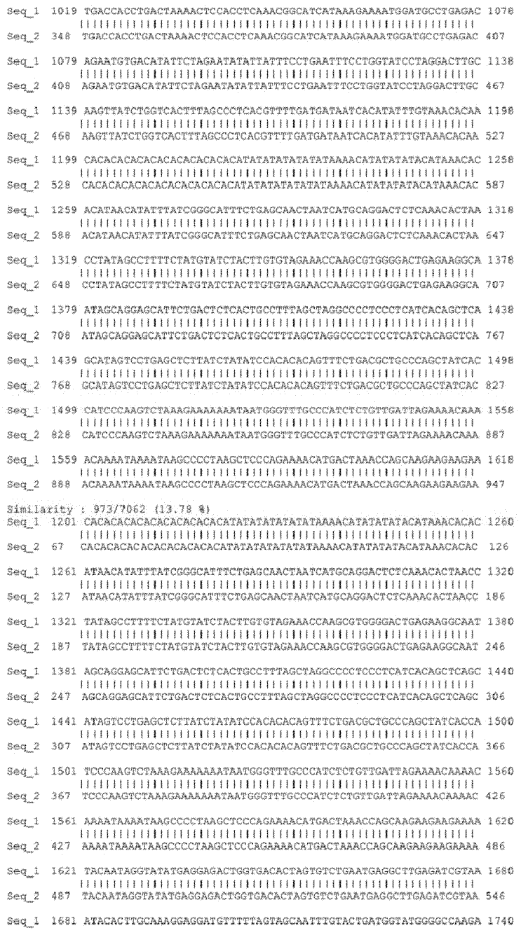

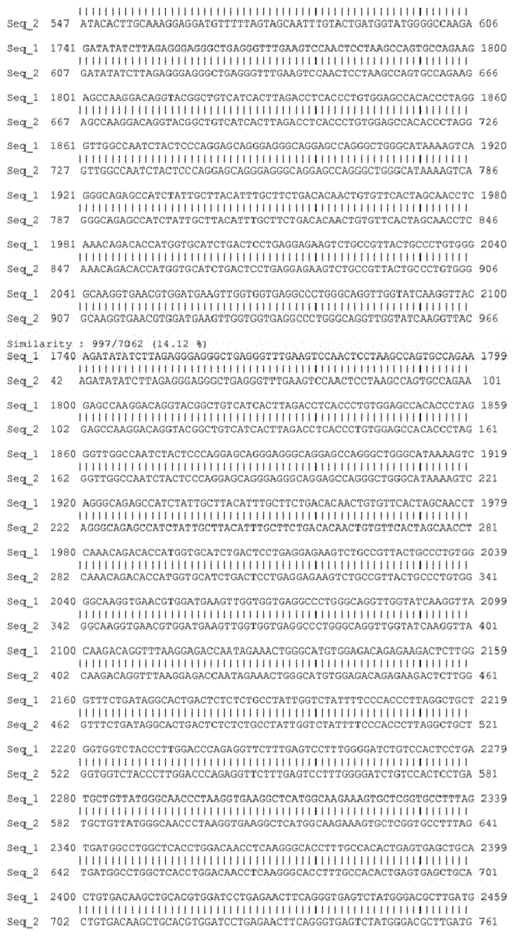

1. Synthesis of reverse β-globin plasmid with HS 1-4: The LCR region of β-globin was predicted on the website http://www.genomatix.de/. According to results of related literatures, HS 1-4 can increase the expression of β-globin, and in most literatures, β-globin is inserted into a recombinant plasmid reversely, which may enhance the expression of β-globin. Therefore, HS 1-4 was ligated to the β-globin sequence in Example 2. Because the recombinant plasmid 8019 constructed in Example 2 included a mutated sequence, a recombinant β-globin gene sequence that had no mutation and included the β-globin LCR HS 4-1, β-globin gene promoter, β-globin coding sequence, and β-globin gene enhancer sequences was designed, which was specifically shown as follows. The sequence was synthesized by Sangon and ligated to a pUC57 vector, and a resulting plasmid was named pHS(4-1)-βp(Exon266 bp)-βglobin-βE-pUC57 and numbered 8023. The plasmid was digested and sequenced. Digestion results of 8023 were shown in FIG. 6A. According to alignment results, the sequence was correct.

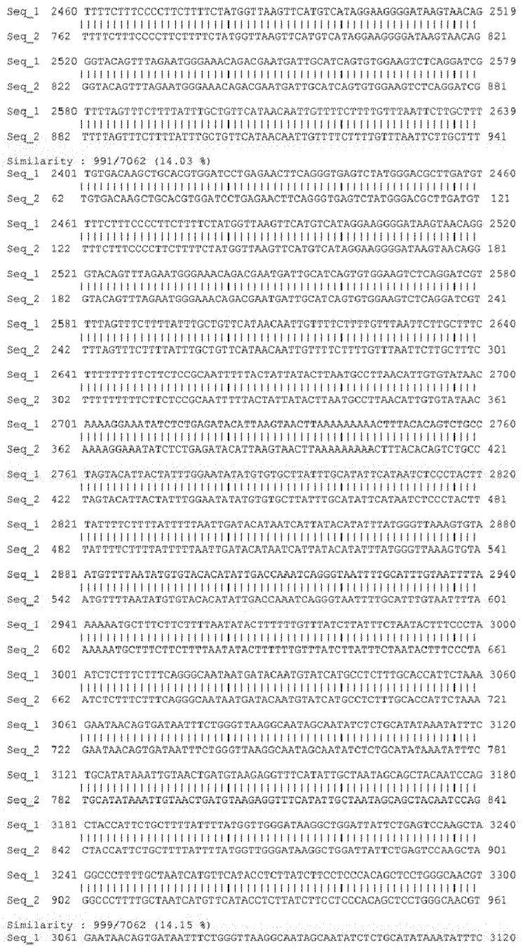

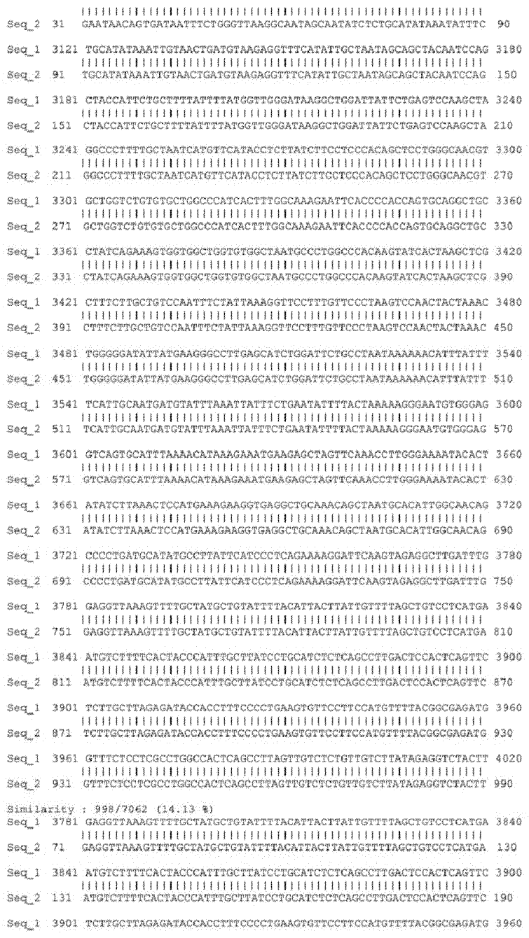

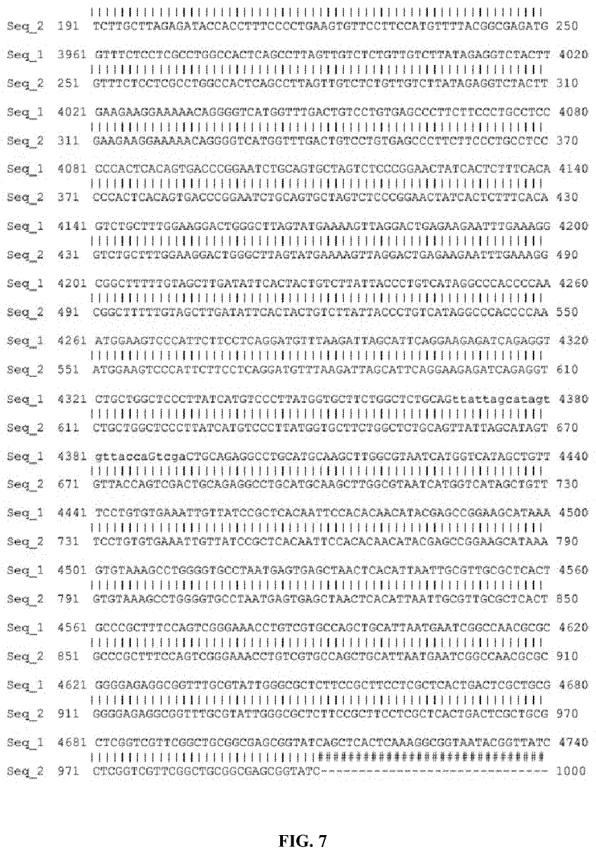

2. A recombinant reverse β-globin plasmid without HS 4 was constructed with 8023 as a template, and the constructed plasmid was named pHS(3-1)-βp(Exon266 bp)-βglobin-βE-pUC57 and numbered 8405. A specific construction method was as follows: a forward primer was designed upstream of HS3, and a KpnI restriction site was added, where the forward primer was HS3-pUC57-F: CTTggtac caagactgagctcagaaga (SEQ ID NO: 16); a reverse primer was designed downstream of the enhancer, and a SalI restriction site was added, where the reverse primer was lv-R-2: GCCGTCGACtggtaacactatgctaataac (SEQ ID NO: 17); PCR was conducted with 8023 as a template, and a product was purified; the PCR product was digested with DpnI, kpnI, and SaII, and the backbone was digested with kpnI and SaII (DpnI Item No.: FD1703, kpnI Item No.: FD 0524, and SaII Item No.: FD0644); and target fragments were recovered by gel extraction, then ligated by T4 DNA ligase, and transformed for amplification. Enzyme digestion and sequencing verification were conducted, and according to enzyme digestion and sequencing verification results, the fragment sequence was correct. The enzyme digestion results were shown in FIG. 6A, the sequencing results were shown in FIG. 7, and detailed experimental methods involved in the construction were the same as above.

3. A recombinant reverse β-globin plasmid without HS 1 was constructed with 8023 as a template, and the constructed plasmid was named pHS(4-2)-βp(Exon266 bp)-βglobin-βE-pUC57 and numbered 8406. A specific construction method was as follows: a forward primer was designed downstream of HS 1, a reverse primer was designed downstream of HS 2, and a BgIII restriction site was added at 5′ end of each of the forward and reverse primers, where the primers were as follows: BgIII-E266-F: GCGagatctATCGTAAATACACTTGC (SEQ ID NO: 18), and BgIII-HS2-R: GCGagatctTTCAGGAAATAATATATTC (SEQ ID NO: 19); PCR was conducted with 8023 as a template, and a product was purified; enzyme digestion was conducted overnight with DpnI and BgIII (BgIII Item No.: FD0084); and target fragments were recovered by gel extraction, then ligated by T4 DNA ligase, and transformed for amplification. KpnI enzyme digestion and sequencing verification were conducted, and according to enzyme digestion and sequencing verification results, the fragment sequence was correct. The enzyme digestion results were shown in FIG. 6A, and detailed experimental methods involved in the construction were the same as above.

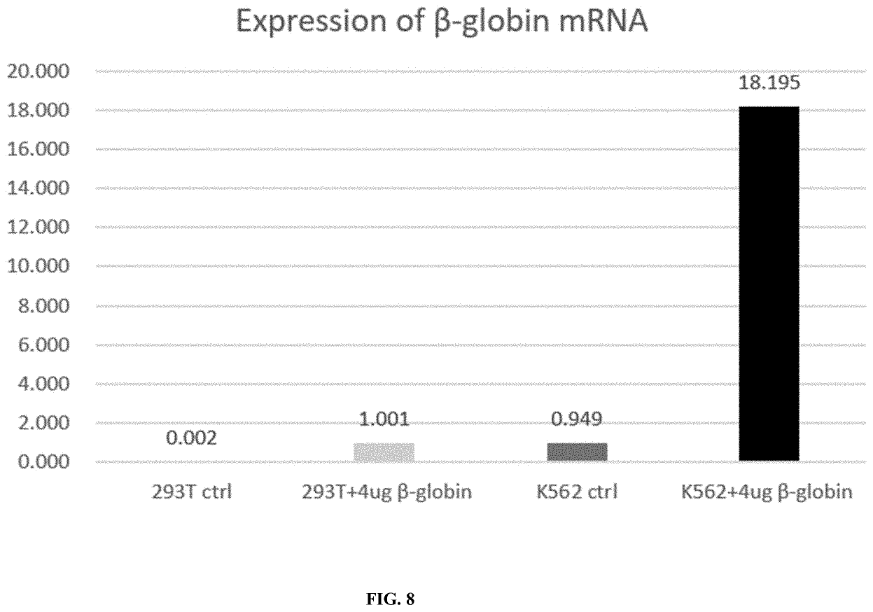

4. K562 and 293T cell lines were each electrotransfected with 4 μg of the plasmid 8023 to verify the specificity of the recombinant plasmid, and a specific method was the same as in Example 2. Results were shown in FIG. 8, and it can be seen that, after transfection of the same β-globin, an expression level of β-globin in 293T cells was still not significantly increased, indicating that the sequence is relatively specific. According to the specificity detection results of the promoter, it is considered that the constructed plasmid has some erythroid specificity.

5. The effectiveness of the above three reverse β-globin sequences with different β-globin LCRs (HS) was detected, and a specific method was as follows:

1) K562 cells were electroporated (electrode tube set, Item No.: No. 1207), and specific steps were the same as in Example 2.

2) Detection of a relative expression level of β-globin mRNA by qPCR: Specific steps were the same as in Example 2.

3) Detection of β-globin level by WB (primary and secondary antibodies for β-globin, Item No.: SC-21757 and 62-6520): Specific steps were the same as in Example 2.

4) Detection results by WB were shown in FIG. 6B and FIG. 6C, and it can be seen that the recombinant plasmid 8405 led to the highest expression level of β-globin, that is, the reverse β-globin plasmid without HS 4 (HS(3-1)-βp(Exon266 bp)-βglobin-βE-puc57) led to the highest expression level of β-globin, indicating that the HS3-1-βp(Exon266 bp)-βglobin-βE sequence can efficiently express β-globin.

It can be seen from the above examples that, in the present disclosure, three human β-globin gene promoter plasmids (promoter-EGFP plasmids) with different lengths were first constructed and optimized, and a promoter region with high activity, strong specificity, and a length of 266 bp was screened out by using the co-transfection method with the Mcherry plasmid. In the present disclosure, with the promoter length screened out, a plasmid without β-globin LCR (HS) is constructed and the effectiveness thereof is tested; then with the promoter length screened out, reverse β-globin plasmids with different β-globin LCRs (HS) are constructed and the effectiveness and specificity thereof are tested; and finally a recombinant sequence with the regulatory region of HS3-1 and the 266 bp promoter and enhancer of the human β-globin gene in sequence is obtained, and the recombinant sequence can efficiently and specifically express the human β-globin, which provides a basis for subsequent gene therapy of thalassemia.

The above descriptions are merely preferred implementations of the present disclosure. It should be noted that those skilled in the art may further make several improvements and modifications without departing from the principle of the present disclosure, but such improvements and modifications should be deemed as falling within the protection scope of the present disclosure.

What is claimed is:

1. A recombinant sequence specifically expressing human β-globin in erythroid cells, wherein the recombinant sequence is human β-globin locus control region (LCR) HS 3-1+human β-globin gene promoter+β-globin gene+β-globin gene enhancer; and
the human β-globin gene promoter is an erythroid-specific human β-globin gene promoter with a nucleotide sequence set forth in SEQ ID NO: 1.

2. The recombinant sequence according to claim 1, wherein a nucleotide sequence of the β-globin gene enhancer is set forth in SEQ ID NO: 2.

3. The recombinant sequence according to claim 1, wherein a nucleotide sequence of the β-globin gene is set forth in SEQ ID NO: 3.

4. The recombinant sequence according to claim 1, wherein the β-globin LCR HS 3-1 comprises β-globin LCR HS3, β-globin LCR HS2, and β-globin LCR HS14 in sequence;
a nucleotide sequence of the β-globin LCR HS1 is set forth in SEQ ID NO: 4;
a nucleotide sequence of the β-globin LCR HS2 is set forth in SEQ ID NO: 5; and
a nucleotide sequence of the β-globin LCR HS3 is set forth in SEQ ID NO: 6.

5. The recombinant sequence according to claim 1, wherein the recombinant sequence comprises at least one selected from the group consisting of the following DNA sequences:
1) a DNA sequence with a nucleotide sequence set forth in SEQ ID NO: 7; and
2) a DNA sequence encoding a recombinant protein with an amino acid sequence set forth in SEQ ID NO: 8.

6. The recombinant sequence according to claim 2, wherein the recombinant sequence comprises at least one selected from the group consisting of the following DNA sequences:
1) a DNA sequence with a nucleotide sequence set forth in SEQ ID NO: 7; and
2) a DNA sequence encoding a recombinant protein with an amino acid sequence set forth in SEQ ID NO: 8.

7. The recombinant sequence according to claim 3, wherein the recombinant sequence comprises at least one selected from the group consisting of the following DNA sequences:
1) a DNA sequence with a nucleotide sequence set forth in SEQ ID NO: 7; and
2) a DNA sequence encoding a recombinant protein with an amino acid sequence set forth in SEQ ID NO: 8.

8. The recombinant sequence according to claim 4, wherein the recombinant sequence comprises at least one selected from the group consisting of the following DNA sequences:
1) a DNA sequence with a nucleotide sequence set forth in SEQ ID NO: 7; and
2) a DNA sequence encoding a recombinant protein with an amino acid sequence set forth in SEQ ID NO: 8.

9. A recombinant vector comprising the recombinant sequence according to claim 1, wherein a backbone vector of the recombinant vector comprises a mammalian expression vector and/or a lentiviral vector.

10. Use of the recombinant sequence according to claim 1 in the preparation of a drug for gene therapy of β-thalassemia.

11. The use according to claim 10, wherein the drug comprising the recombinant sequence, or a recombinant vector, and
wherein the recombinant vector comprises the recombinant sequence, and a backbone vector of the recombinant vector comprises a mammalian expression vector and/or a lentiviral vector.


SEQUENCE LISTING (19 sequences)

SEQ ID NO: 1 - DNA, 268 bp, Artificial Sequence
  erythroid-specific human beta-globin gene promoter
atcgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgctt

SEQ ID NO: 2 - DNA, 279 bp, Artificial Sequence
  beta-globin gene enhancer
tgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttacca

SEQ ID NO: 3 - DNA, 2167 bp, Artificial Sequence
  beta-globin gene
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcag

SEQ ID NO: 4 - DNA, 558 bp, Artificial Sequence
  beta-globin LCR HS1
tttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgag

SEQ ID NO: 5 - DNA, 470 bp, Artificial Sequence
  beta-globin LCR HS2
gctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaa

SEQ ID NO: 6 - DNA, 644 bp, Artificial Sequence
  beta-globin LCR HS3
aagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattat

SEQ ID NO: 7 - DNA, 4386 bp, Artificial Sequence
  recombinant sequence
aagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagatcgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgcctcccccactcacagtgacccggaatctgcagtgctagtctcccggaactatcactctttcacagtctgctttggaaggactgggcttagtatgaaaagttaggactgagaagaatttgaaaggcggctttttgtagcttgatattcactactgtcttattaccctgtcataggcccaccccaaatggaagtcccattcttcctcaggatgtttaagattagcattcaggaagagatcagaggtctgctggctcccttatcatgtcccttatggtgcttctggctctgcagttattagcatagtgttacca

SEQ ID NO: 8 - protein, 147 aa, Artificial Sequence
  recombinant protein
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 9 - DNA, 28 bp, Artificial Sequence
  Primer E152 FP
cgattaatagtgccagaagagccaagga

SEQ ID NO: 10 - DNA, 31 bp, Artificial Sequence
  Primer E266 FP
cgattaatcgtaaatacacttgcaaaggagg

SEQ ID NO: 11 - DNA, 28 bp, Artificial Sequence
  Primer E422 FP
cgattaatctggagacgcaggaagagat

SEQ ID NO: 12 - DNA, 31 bp, Artificial Sequence
  Primer Exon-RP
atattgctagcaagcaatagatggctctgcc

SEQ ID NO: 13 - DNA, 30 bp, Artificial Sequence
  Primer enhancer-R
atgcggccgctggtaacactatgctaataa

SEQ ID NO: 14 - DNA, 21 bp, Artificial Sequence
  forward primer mRNA-F-2
ctgaggagaagtctgccgtta

SEQ ID NO: 15 - DNA, 20 bp, Artificial Sequence
  reverse primer mRNA-R-2
gaggttgtccaggtgagcca

SEQ ID NO: 16 - DNA, 27 bp, Artificial Sequence
  forward primer HS3-pUC57-F
cttggtaccaagactgagctcagaaga

SEQ ID NO: 17 - DNA, 30 bp, Artificial Sequence
  reverse primer lv-R-2
gccgtcgactggtaacactatgctaataac

SEQ ID NO: 18 - DNA, 26 bp, Artificial Sequence
  Primer BgIII-E266-F
gcgagatctatcgtaaatacacttgc

SEQ ID NO: 19 - DNA, 28 bp, Artificial Sequence
  Primer BgIII-HS2-R
gcgagatctttcaggaaataatatattc